(19) United States
(12) Patent Application Publication
(10) Pub. No.: US 2012/0184931 A1
(43) Pub. Date: Jul. 19, 2012

(54) Suction line for a vacuum wound treatment device comprising connector parts

(76) Inventor: Denny Horn, Freiburg (DE)

(21) Appl. No.: 13/347,747

(22) Filed: Jan. 11, 2012

Related U.S. Application Data
(60) Provisional application No. 61/434,020, filed on Jan. 19, 2011.

(30) Foreign Application Priority Data
Jan. 14, 2011 (DE) 10 2011 009 241.2

(51) Int. Cl. A61M 1/00

(52) U.S. Cl. 604/319

(57) ABSTRACT

A suction line (8) for a vacuum wound treatment device (2) has a first (14) and second suction line section (16) connected together by a first connector part (18) borne on the first suction line section (14) and a second connector part (20) borne on the second line section (16). A connector part (18) has a valve device (40, 41) with a valve body (42, 43) that closes the flow cross-section of a connector part (18) in the uncoupled state of the connector parts (18, 20) and which is structured to completely open a smallest flow-cross-section (54, 56) when a line section (44, 46) of one connector part (20), which forms a flow channel (26, 28), moves against and displaces the valve body (42, 43) during assembly of the connector parts (18, 20).

This application claims benefit of 61/434,020 filed Jan. 19, 2011 as well as Paris Convention priority of DE 10 2011 009 241.2 filed Jan. 14, 2011, the entire disclosures of which are hereby incorporated by reference.

### BACKGROUND OF THE INVENTION

The invention concerns a suction line for a vacuum wound treatment device, the suction line comprising a first suction line section and a second suction line section, wherein the first suction line section leads away from a vacuum-tight wound cover and bears a first connector part at its end facing away from the wound, which connector part delimits at least one first flow channel, and wherein the second suction line section bears a second connector part, which delimits at least one second flow channel, is designed to be complementary to the first connector part, forms with the latter a vacuum-tight detachable connection assembly, and continues from there towards a vacuum-generating unit, wherein a connector part has a valve device which is mechanically opened during assembly of the connector parts, and is automatically closed when the connector parts are pulled apart to thereby prevent fluid medium from dripping out of the first suction line section when the connector parts are released from each other after vacuum operation.

Vacuum wound treatment devices of the above-mentioned type have been known for a long time and are increasingly used in the medical field for treating wounds the healing process of which is problematic. There are conventional suction lines for vacuum communication between a wound space that is substantially sealed in a vacuum-tight fashion by the wound cover and a vacuum-generating unit, which are provided with several sections that can be coupled by means of connector parts, see e.g. WO 2010/003156 A1. The document US 2007/0169825 A1 already discloses a suction line with connector parts comprising the above-mentioned features. Each connector part has a valve device of extremely complex design with a barrel-shaped rotatable valve body having a through-hole, wherein the barrel-shaped valve body is rotated back and forth during assembly or release of the connector parts through very complex mechanical coupling in such a fashion that a flow communication is obtained in the assembled state and blocking is obtained when the connector parts are separated.

It is the underlying purpose of the present invention to produce a detachable connection assembly for a suction line of a vacuum wound treatment device of the above-mentioned type, the construction of which is considerably facilitated compared to the above-mentioned device and the production of which is therefore also more economical.

### SUMMARY OF THE INVENTION

In accordance with the invention, this object is achieved with a suction line of the above-mentioned type in that the valve device of the connector parts is designed in such a fashion that a valve body, which closes the flow cross-section of a connector part in the uncoupled state of the connector parts, is completely displaced from a smallest flow cross-section of the first and second flow channels during assembly of the connector parts and therefore completely opens this smallest flow-cross-section in that a line section, forming the flow channel, of one connector part moves against the valve body during assembly of the connector parts, thereby displacing the valve body.

The invention therefore proposes to design the connector parts and the valve device thereof in such a fashion that, during assembly of the connector parts, the valve body is subjected to a simple displacement motion by the line section of one connector part. It is therefore not necessary to design a complex mechanical transmission connection, which, starting from a translatory assembly motion, realizes a rotary motion of the valve body, since the respective line section of one connector part moves against the valve body in a simple and reliable fashion during assembly, thereby displacing the valve body. In accordance with the invention, the valve body is thereby completely displaced from the smallest flow cross-section both of the first and the second flow channels. This aims to ensure that the valve device does not entail any further restriction of the flow cross-section in the area of the connector parts but that the flow cross-section is determined only by an already predetermined minimum flow cross-section of the respective first and second flow channels of the connector parts in the open state of the valve device.

Due to the simple design of the valve device, an embodiment can be realized, in which the suction line and both connector parts are designed to have at least two lumens. For this reason, they comprise at least two flow channels that lead away from the wound cover and are guided through both connector parts.

It has also turned out to be advantageous when the two connector parts can be positively coupled to each other by moving one engaging-behind element of one part into a position in which it engages behind the other part when the vacuum-tight detachable connection assembly is established.

In a further development of this idea, the positive coupling of the connector parts is advantageously automatically established during assembly of the connector parts in that an engaging-behind element of one connector part moves against an engagement slope of the other connector part, is thereby deflected and is finally locked in a position in which it engages behind the other part.

In a further development of the invention, the valve body of the valve device is designed in the form of lamellas or lips. In this case, the valve body can be displaced without much resistance. A valve body designed in the form of lamellas or lips can moreover be formed and designed in a three-dimensional fashion such that it provides effective protection against leakage or dripping of liquid.

In a particularly important further development of this inventive idea, the valve body is formed on an insertion part that is inserted into one of the connector parts and delimits a flow channel. In accordance with this inventive idea, the valve body cannot be loosely displaced back and forth but is itself a component of an insertion part that is stationarily provided on the respective connector part.

In a further design of this inventive idea, the insertion part is inserted into a depression of one connector part, wherein the depression extends in the assembly direction of the connector parts and surrounds a flow channel, and a male connector section of the other connector part is inserted into this depression during assembly of the connector parts, moves against the valve body and displaces the valve body from the flow cross-section.

The respective valve body, which is designed, in particular, in the form of lamellas or lips, moreover advantageously obtains its restoring force for closing the respective flow channel from its own elasticity and connection to the insertion part, which is easy and economical to realize. In this connection, it has turned out to be advantageous when the valve device does not necessarily have to seal the vacuum to 100%, since, after separation of the suction line sections, the vacuum in the area of the wound is anyway released after a relatively short time due to leakage of air coming in from the outside. It is rather essential to effectively prevent dripping in the area of the connector parts and, in particular, in the area of the first connector part on the wound side. Exactly this can be achieved with connector parts of the inventive design.

The invention further proposes to design the insertion part comprising the valve body in such a fashion that it has a rotationally symmetrical section, the outer side of which can be radially disposed against the respective connector part. This rotationally symmetrical, in particular, cylindrical or slightly conical outer configuration facilitates insertion of the insertion part into a typically cylindrical or slightly conical depression of the respective connector part.

It has also turned out to be advantageous for the lamella-like or lip-like valve body to be formed radially inside of this rotationally symmetrical section. In a further development of this inventive idea, it has turned out to be advantageous for the lamella-like or lip-like valve body to be formed by wall sections of the insertion part, which extend in the form of wedges with respect to one another. The wedge-shaped wall sections may thereby advantageously extend at an angle with respect to the flow direction of the flow channels. Their orientation and wall thickness are thereby advantageously selected such that they can be displaced from the flow cross-section, thereby being slightly plastically deformed or compressed.

In the closed state of the valve device, the lamella-like or lip-like valve bodies may substantially at least almost abut each other via a separating gap.

The wall sections extending in the form of wedges with respect to one another and the insertion part are all advantageously designed in such a fashion that, in the blocked state of the valve device, receiving pockets for residual liquid are formed in the area of the line wall of the respective flow channel, which terminate, in particular, in acute angles.

The insertion part may advantageously be an injection-molded plastic part.

The invention moreover also comprises a connector provided with a valve device in accordance with the invention, comprising a first and a second connector part as described above and in the claims.

Further features, details and advantages of the invention can be extracted from the attached claims and the drawing and the following description of a preferred embodiment of the invention.

### BRIEF DESCRIPTION OF THE DRAWING

FIG. 1 shows a schematic view of a vacuum wound treatment device with wound cover, suction line, liquid absorbing container and vacuum-generating device;

FIG. 2 shows a top view of a first and a second connector part which are assembled for generating a flow communication;

FIG. 3 shows a view in the direction of arrow III of FIG. 2;

FIG. 4 shows a sectional view of the assembled connector parts according to FIG. 1;

FIGS. 5 and 6 show enlarged, partial, sectional views of the connector parts according to FIGS. 2 to 4 during assembly and release from each other; and

FIGS. 7a through d show different views and partial views of a component of a valve device of the connectors in accordance with FIGS. 2 to 6.

### DESCRIPTION OF THE PREFERRED EMBODIMENT

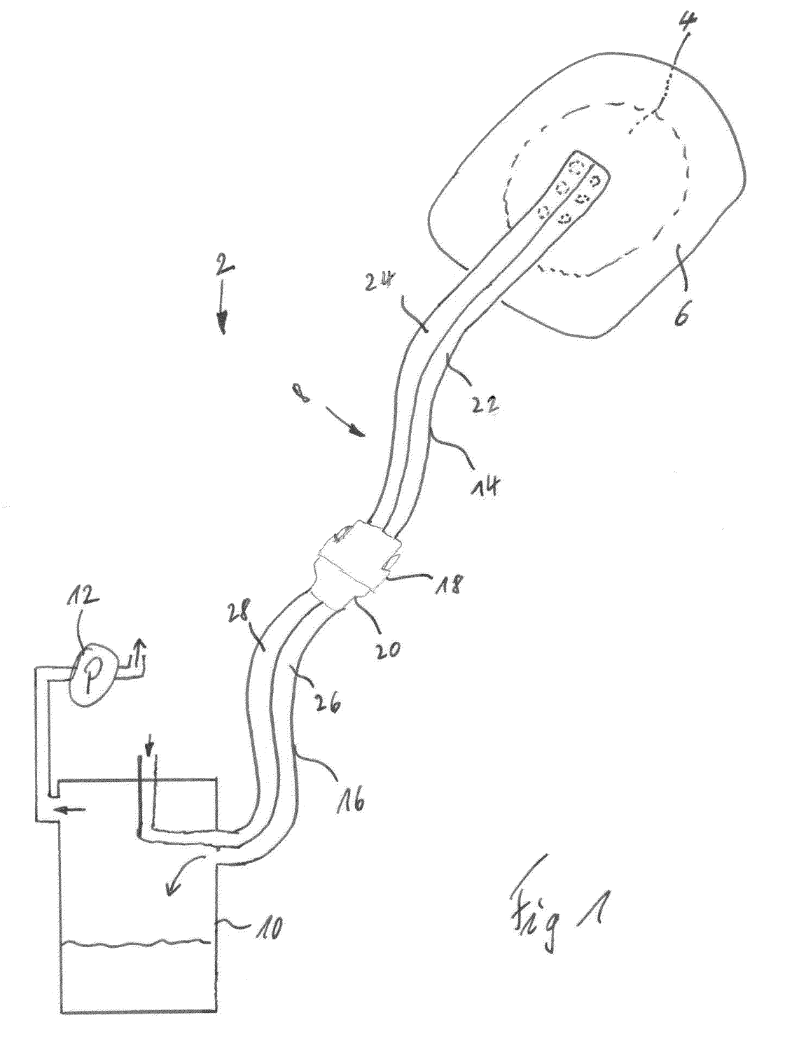

FIG. 1 schematically shows a vacuum wound treatment device designated in total by reference numeral 2, comprising a wound cover 6 that seals a wound space 4 in a substantially vacuum-tight fashion, a suction line 8, a liquid absorbing container 10, and a vacuum-generating device 12. The wound cover 6 communicates via the suction line 8 with the liquid absorbing container 10 that can be evacuated such that a vacuum can be applied to the wound space 4 in a conventional fashion and wound fluid can be discharged from the wound space 4 via the suction line 8 and can be collected in the liquid absorbing container 10. The suction line 8 comprises a first suction line section 14 on the wound side and a second subsequent second suction line section 16 which leads to the liquid absorbing container 10, wherein the suction line sections can be connected to each other or be released from each other via a first connector part 18 on the first suction line section and a second connector part 20 on the second suction line section 16. In the illustrated example, the first suction line section 14, the second suction line section 16 and also the two connector parts 18, 20 are formed to have two lumens, i.e. each comprises two parallel flow channels 22, 24 and 26, 28. One flow channel 22, 26 having a slightly larger flow cross-section is preferably used as a vacuum and drainage channel, whereas the other flow channel 24, 28 is used as rinsing channel for controlled supply of e.g. air. FIGS. 2 to 6 illustrate the two connector parts 18, 20, their components, and the interaction between them.

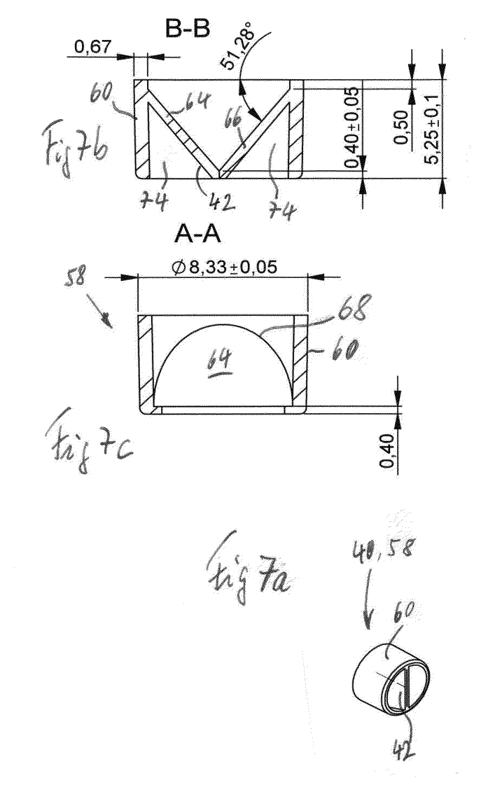

The connector parts 18, 20 can be positively coupled to each other. In the illustrated example, the second connector part 20 comprises e.g. two engaging-behind elements 30 designed as engagement hooks, which are moved against a sloped engagement wall 32 of the other connector part during assembly of the connector parts 18, 20, are thereby deflected and are finally locked in an engaged-behind position in a window-like recess 34 of the other connector part 18. The assembly process is illustrated in FIG. 5 and the assembled state is illustrated in FIG. 4.

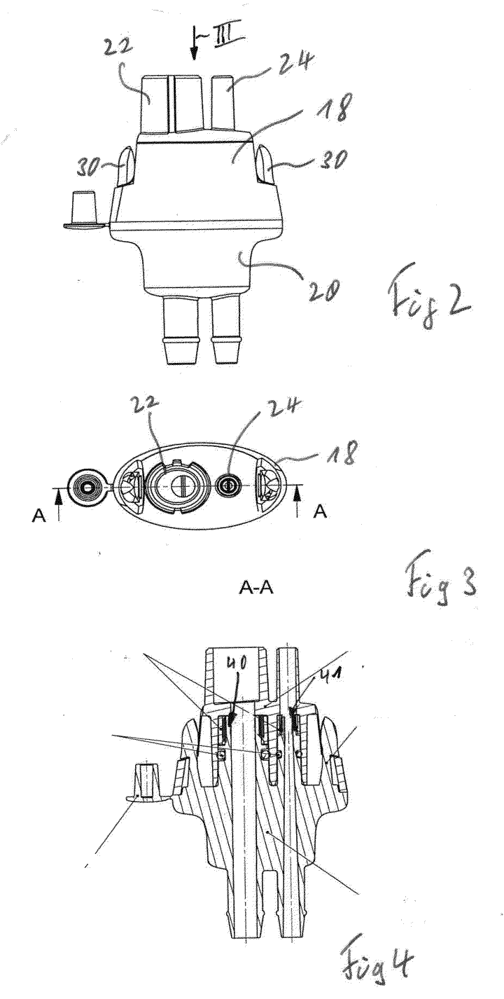

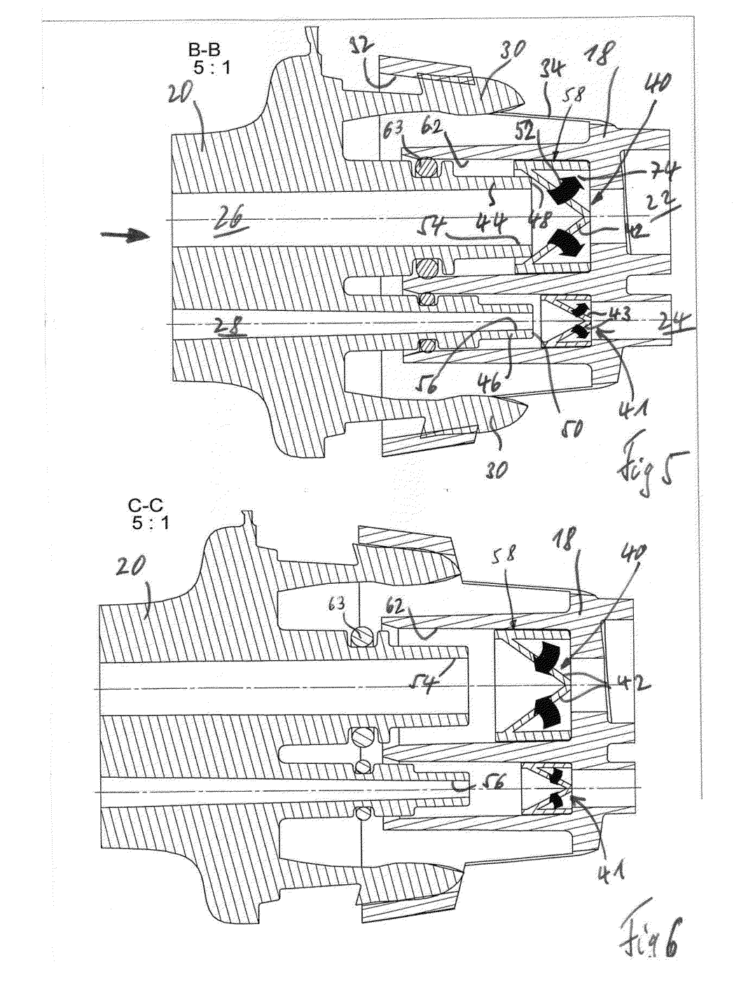

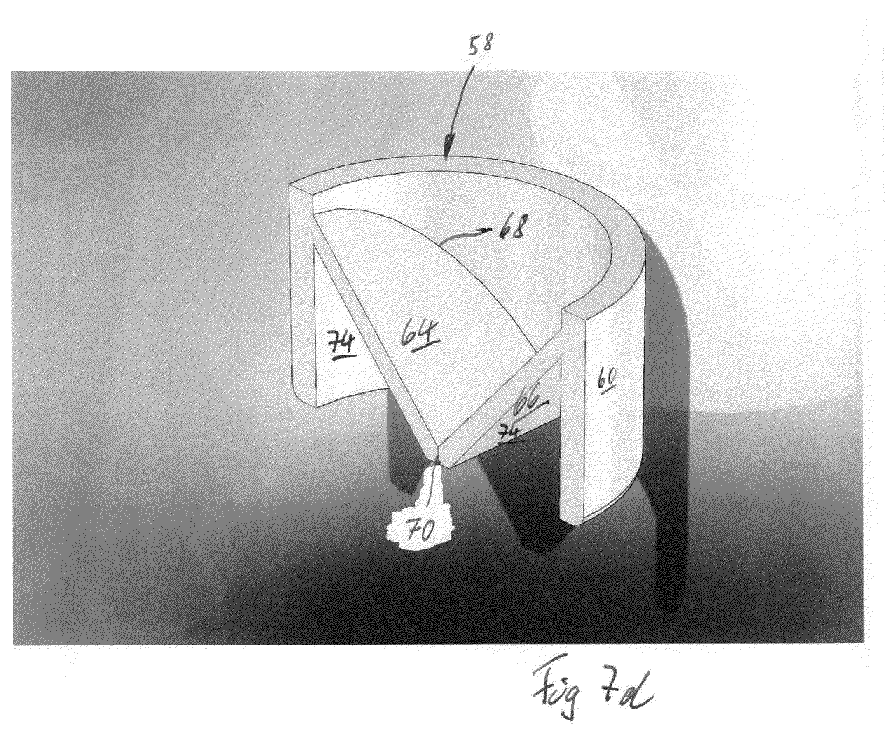

In the assembled state of the connector parts 18, 20, the first flow channels 22, 24 that continue in the connector parts, are flow-connected to the second channels 26, 28 such that vacuum or flow communication is possible. Each valve device designated by reference numerals 40, 41 is then in a position opening the respective flow path as is illustrated in FIG. 4. A lamella-like or lip-like valve body 42, 43 is thereby displaced from the position illustrated in FIGS. 5 and 6, in which the flow channel is closed, to the position illustrated in FIG. 3. This displacement is realized in that a line section 44, 46 delimiting the respective second flow channel 26, 28 moves with its respective end face 48, 50 against the respective lamella-like or lip-like valve bodies 42, 43, thereby displacing them in the direction of arrows 52. The valve bodies 42, 43 are thereby displaced in such a fashion that they are completely displaced from a smallest flow cross-section 54 or 56 of the connector parts 18, 20 in the area of the valve device 40, 41. In other words, this means that the valve bodies 42, 43 of the respective valve device 40, 41 do not limit the flow cross-section of the first and second flow channels 22, 24, or 26, 28 in the assembled state of the connector parts 18, 20. This is particularly advantageous since there is often the danger that deposits, crusts or the like form in the area of intersections, interlocking engagements and material transitions, which are typically present at coupling points of flow connections and often cause blockages. This danger is further minimized in that the respective valve bodies 42, 43 of the valve devices 40, 41 are completely displaced from the flow cross-section.

The respective valve device 40, 41 comprises an insertion part 58, which is illustrated in FIGS. 7a to d and is designed as injection-molded part, the outer periphery of which is formed by a rotationally symmetrical section 60 in the embodiment given by way of example.

The respective insertion part 58 can be inserted into a cylindrical or, like in the example, slightly conically tapering depression 62 of the first connector part 18. It is thereby secured by clamping. In this case, the depression 62 has a female design and surrounds the respective first flow channel 22, 24 of the first connector part 18. It receives the male line section 44, 46 as intended, the male line section delimiting the respective second flow channel 26, 28 of the second connector part 20. O-ring shaped sealing elements 63 are moreover provided for sealing.

The above-mentioned lamella-like or lip-like valve body 42, 43 is formed radially inside of the rotationally symmetrical section 60 of the insertion part 58. Each valve body comprises two wall sections 64, 66 that are integrally formed on the section 60. In the example, the connection of the wall sections 64, 66 to the rotationally symmetrical section 60 follows a parabolic path 68, which is optimally illustrated in FIG. 7d. In the blocked state of the valve device 40, 41, the lamella-like or sealing lip-like wall sections 64, 66 substantially at least almost abut each other via a separating gap 70 that extends in a linear fashion in this example. This is illustrated in FIG. 5 prior to assembly of the connector parts 18, 20 or in FIG. 6 during pulling apart of the connector parts. The lamella-like or lip-like wall sections 64, 66 of the insertion part 58 move, starting from their open position (FIG. 4), into their closed position illustrated in FIGS. 7a to d due to a resilient restoring force that results from the connection of the wall sections 64, 66 to the rotationally symmetrical section 60 of the insertion part 58.

In this closed position, the valve device 40, 41 forms a receiving pocket 74 for residual liquid, which preferably and by way of example terminates in acute angles and is formed by the inclined wall sections 64, 66 and the inner side of the rotationally symmetrical section 60 of the insertion part 58.

I claim:

1. A suction line for a vacuum wound treatment device, the vacuum wound treatment device having a vacuum-tight wound cover and a vacuum-generating unit, the suction line comprising:
a first suction line section leading away from the vacuum-tight wound cover;
a second suction line section leading towards the vacuum generating unit;
a first connector part borne in said first suction line section at an end thereof facing away from a wound, said first connector part delimiting at least one first flow channel;
a second connector part borne in said second suction line section, said second connector part having a line section element delimiting at least one second flow channel, wherein said second connector part is structurally complementary to said first connector part, said first and said second connector parts thereby cooperating to form a vacuum-tight detachable connection assembly; and
a valve device disposed in said first connector part, said valve device having a valve body which is mechanically opened during assembly of said first and said second connector parts and which is automatically closed when said first and said second connector parts are pulled apart, thereby closing a flow cross-section of said first connector part to prevent fluid medium from dripping out of the first suction line section following vacuum operation, wherein, during assembly of said first and said second connector parts, said line section element of said second connector part moves against and completely displaces said valve body away from a smallest flow cross-section of said first and said second flow channels to completely open said smallest flow-cross-section.

2. The suction line of claim 1, wherein said first suction line section, said second section line section and said first and said second connector parts, each have at least two lumens.

3. The suction line of claim 1, wherein said first and said second connector parts are positively coupled to each other by moving an engaging-behind element of one part into a position in which that element engages behind an other part when a vacuum-tight, detachable connection assembly is established.

4. The suction line of claim 3, wherein a positive coupling of said first and said second connector parts is automatically established during assembly thereof in that an engaging-behind element of one connector part moves against an engagement slope of an other connector part, is thereby deflected and is finally locked in a position engaging behind said other part.

5. The suction line of claim 1, wherein said valve body is designed in a form of lamellas or lips.

6. The suction line of claim 5, wherein said valve body is formed on an insertion part that is inserted into said first connector part and surrounds said first flow channel.

7. The suction line of claim 6, wherein said insertion part is inserted into a depression of said first connector part, said depression extending in an assembly direction of said first and said second connector parts and surrounding said first flow channel, wherein said line section element of said second connector part comprises a male connector section which is inserted into said depression during assembly of said first and said second connector parts, is moved against said valve body, and displaces said valve body away from said flow cross-section.

8. The suction line of claim 6, wherein said valve body obtains, from an inherent thereof elasticity and via connection to said insertion part, a restoring force for closing a respective flow channel.

9. The suction line of claim 6, wherein said insertion part has a rotationally symmetrical section, a radially outer side of which lies against said first connector part.

10. The suction line of claim 9, wherein said lamellas or lips of said valve body are formed radially inside of said rotationally symmetrical section.

11. The suction line of claim 6, wherein said lamellas or lips of said valve body are formed by wall sections of said insertion part, which extend in a form of wedges with respect to one another.

12. The suction line of claim 11, wherein said wall sections of said insertion part, which extend in said form of wedges with respect to one another, extend at an angle with respect to a flow direction of said first flow channel.

13. The suction line of claim 11, wherein said wall sections of said insertion part, which extend in said form of wedges with respect to one another, substantially abut each other via a separating gap.

14. The suction line of claim 13, wherein said separating gap extends in a linear fashion.

15. The suction line of claim 11, wherein said wall sections of said insertion part, which extend in said form of wedges with respect to one another, and said insertion part are designed in such a fashion that, in a blocked state of the valve device, receiving pockets for residual liquid are formed, which terminate in acute angles.

16. The suction line of claim 6, wherein said insertion part is an injection-molded part.
